SEQUENCE LISTING (11 sequences)

SEQ ID NO: 1 - DNA, 23 bp, Artificial
  Synthetic oligonucleotide probe
tagaaaggatatgacttatctca

SEQ ID NO: 2 - DNA, 22 bp, Artificial
  Synthetic oligonucleotide probe
tagaaagatatgacttatctca

SEQ ID NO: 3 - DNA, 19 bp, Artificial
  Synthetic oligonucleotide probe
aaaggatatgacttatctc

SEQ ID NO: 4 - DNA, 22 bp, Artificial
  Synthetic oligonucleotide probe
ctgcgtagcgaattaatatgac

SEQ ID NO: 5 - DNA, 19 bp, Artificial
  Synthetic oligonucleotide probe
aaagaatatctctgatctc

SEQ ID NO: 6 - DNA, 34 bp, Artificial
  Synthetic oligonucleotide probe
ggaggagggctgctgcttgaggaagtataagaat

SEQ ID NO: 7 - DNA, 21 bp, Artificial
  Synthetic oligonucleotide probe
cgcttgatgactcagccggaa

SEQ ID NO: 8 - DNA, 22 bp, Artificial
  Synthetic oligonucleotide probe
tagaaaggatatgacttatctc

SEQ ID NO: 9 - DNA, 20 bp, Artificial
  Synthetic oligonucleotide probe
aaaggatatgacttatctca

SEQ ID NO: 10 - DNA, 23 bp, Artificial
  Synthetic oligonucleotide probe
tagaaaggatatgacttatctca

SEQ ID NO: 11 - DNA, 38 bp, Artificial
  Synthetic oligonucleotide probe
aaaggatatgacttatctcaaaggatatgacttatctc

(19) United States
(12) Patent Application Publication
(10) Pub. No.: US 2008/0233644 A1
(43) Pub. Date: Sep. 25, 2008

(54) Chimeric Transcription Factor Decoy Oligonucleotides

(76) Inventors: Helen Fillmore, Richmond, VA (US); Jessicca I. Martin Rege, Lynnwood, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 10/570,160

(22) PCT Filed: Sep. 10, 2004
(86) PCT No.: PCT/US04/29465

§ 371 Date: Jan. 17, 2008

Related U.S. Application Data
(60) Provisional application No. 60/502,285, filed on Sep. 12, 2003.

(51) Int. Cl. C12N 5/02; C07H 21/00

(52) U.S. Cl. 435/375; 536/24.5

(57) ABSTRACT

A method for the treatment of the brain cancer glioblastoma multiforme (GBM) is provided. The method involves decreasing the expression of Matrix Metalloproteinase-1 (MMP-1) expression by providing transcription factor decoy nucleotides that mimic single nucleotide polymorphisms responsible for MMP-1 overexpression.

### BACKGROUND OF THE INVENTION

1. Field of the Invention

The invention generally relates to the treatment of diseases caused or exacerbated by the over-expression of Matrix Metalloproteinase-1 (MMP-1), such as glioblastoma multiforme (GBM) cancer. In particular, the invention provides a method of decreasing Matrix Metalloproteinase-1 (MMP-1) expression by providing chimeric transcription factor decoy oligonucleotides that mimic a specific region containing single nucleotide polymorphisms responsible for MMP-1 over-expression.

2. Background of the Invention

Despite many advances in therapeutic and surgical techniques for glioblastoma multiforme (GBM), this form of brain cancer still remains incurable. A hallmark of GBM is the ability of the glioma cells to aggressively infiltrate surrounding brain tissue. The invasive nature of glioma cells is a key challenge in considering treatment for patients with GBM.

Certain members of the matrix metalloproteinase (MMP) family have been shown to play a role in tumor cell invasion and metastasis. For example, a single nucleotide polymorphism (SNP) at position −1607 in the Matrix Metalloproteinase-1 (MMP-1) promoter DNA, resulting from the insertion of a guanine nucleotide next to a pre-existing single guanine nucleotide (1G) to form a 2G allele, creates a functional eukaryotic transcription factor (TF) binding site (Ets-1). The presence of this variant 2G allele is associated with significantly higher transcription of MMP-1 from its promoter. In several published studies, the incidence of the 2G allele has been shown to be significantly higher in aggressive and metastatic tumors. In addition, an adjacent activating protein 1 (AP1) site at −1602 has been shown to cooperate with the Ets binding site created by the SNP. Ets-1, further aiding in transcriptional activation of MMP-1. It has previously been reported that there is a significant increase in the 2G MMP-1 genotype in glioblastomas (p<0.02) when compared to published normal distributions. In addition, in glioma cells presence of the 2G SNP variant correlates with increases in MMP-1 transcriptional activity.

There exists an ongoing need to develop new methods of treating aggressive metastatic cancers and invasive cancers such as GBMs, the prior art having thus far failed to provide such methodology. In particular, the prior art has failed to provide methods for attenuating over-expression of MMP-1, especially when due to the presence of the 2G allele.

### SUMMARY OF THE INVENTION

The present invention provides chimeric “decoy” oligodeoxynucleotides (ODNs) that are designed to mimic at least two TF binding sites within an MMP promoter, particularly the Ets-1 and Ap-1 DNA binding sites of the MMP-1 promoter, and methods of their use. The chimeric decoys are double-stranded. The decoy ODNs are administered to cells either therapeutically to attenuate or prophylactically to prevent, the over-expression of MMPs such as MM-1. In one embodiment, the decoy ODNs contain SEQ IN NO: 3 alone or in combination with additional flanking sequences as illustrated by SEQ ID NOS: 8-10. In other embodiments, the chimeric decoy ODNs may contain multiple copies of one or more binding sites, as illustrated by SEQ ID NO:11. By reducing or inhibiting transcription of MMP proteins, potentially deleterious effects of the expression (and particularly the over-expression) of an MMP protein can be prevented, or attenuated, either partially or completely. In preferred embodiments, the cells are cancer cells or cells that are likely to become cancerous, particularly glioma cells such as those involved in the brain cancer glioblastoma multiforme (GBM).

### BRIEF DESCRIPTION OF THE DRAWINGS

FIG. 1. Increase in transcriptional activity in glioblastoma cells after transfection with the 2G promoter or the 1G promoter.

FIG. 2. Nuclear extracts isolated from U87 and T98 glioma cells bind to both the 1G and 2G promoter probes. This figure shows a summary of relative band intensities compared to free probe.

FIGS. 3A and B. Designed decoy interferes with T98 nuclear proteins binding to MMP-1 2G promoter probe, and not the 1G promoter probe. Competitive EMSA relative band intensities of upper band of A, 1G promoter and B, 2G promoter. A decrease in band intensity is seen in both 1G and 2G promoter probes with decoy treatment. Similar results are seen in U87 cells (data not shown).

FIG. 4. Variant chimeric decoys based on SEQ ID NO:3.

### DETAILED DESCRIPTION OF THE PREFERRED EMBODIMENTS OF THE INVENTION

The present invention provides methods of inhibiting or reducing expression of Matrix metalloproteinases (MMPs), particularly the metastasis-associated protein Matrix Metalloproteinase-1 (MMP-1). The methods involve introducing oligonucleotide (ODN) “decoys” into cells that over-express the MMP protein. The decoy ODNs compete with eukaryotic transcription factor (TF) binding sites on the MMP promoter for the binding of TFs. This competition “ties up” the T's so that none of them are available or fewer of them are available to bind to bona fide promoter TF binding sites on the cellular DNA, thus decreasing the expression of the MMP protein. In a preferred embodiment, decoy ODNs are designed to mimic the DNA that encodes the SNP at position −1607 in the Matrix Metalloproteinase-1 (MMP-1) promoter (i.e. the 2G allele) which is an Ets-1 binding site.

By “inhibiting or reducing expression of MMPs” we mean that the level of the MMP that is expressed in a cell is reduced by at least about 40%, and preferably by at least about 100% compared to the level of MMP that is expressed in comparable cells that are not treated with the decoy ONTs.

In general, the decoys of the present invention are used to decrease or prevent expression of MMPs that are over-expressed. By “over-expressed” we mean that the level of MMP expressed by the cell is higher than the level in a normal cell counterpart. However, those of skill in the art will recognize that the decoy ONTs may also be utilized to decrease expression of MMPs in cells in which MMPs are expressed at a normal level.

In a preferred embodiment of the invention, the MMP whose expression is inhibited is MMP-1. However, other MMPs exist, the expression of which may also be inhibited by the methods of the present invention, including but not limited to MMP-3, and others.

Those of skill in the art will recognize that several means exist by which cells that over-express an MMP or that have the potential to over-express an MMP may be identified. In general, over-expression of an MMP can be detected by measuring expression of the protein directly by known methods (e.g. by using antibodies to the MMP, ELISAs, measuring transcription message levels, etc).

The decoy ODNs of the present invention will, in general, range from about 19 to about 30 or more deoxynucleotides in length, and will preferably range from about 19 to about 22 nucleotides in length. The DNA sequence that makes up a chimeric decoy DNA includes at least two transcription factor DNA binding motifs in order to promote specificity (i.e. they are “chimeric”). This is in contrast to previous decoys which contain only one binding motif. The sequence of a decoy may mimic closely or exactly the sequence of a known MMP TF binding site; the sequence may include a consensus sequence for MMP transcription factor binding; the chimeric decoy sequence may include other motifs known to promote TF binding such as AP-1, Ets-1, and the like; the chimeric decoy may include sequences that flank the TF sites that include specific DNA of the target gene.

In one embodiment of the invention, the chimeric decoy ODN is represented by SEQ ID NO:3 (that is, the chimeric decoy ODN includes only the DNA set forth in SEQ ID NO:3). However, the invention also encompasses variants of SEQ ID NO:3 that contain conservative nucleotide substitutions. By a “conservative substitution”, we mean a substitution of one nucleotide by another that maintains the ability of the oligonucleotide to perform its intended function, e.g. to bind a targeted transcription factor. Variants of SEQ ID NO:3 may contain additional extensions of the chimeric decoy by adding either to the 5′ or 3′ (or both) specific nucleotides that match the targeted DNA, as illustrated in FIG. 5, where SEQ ID NOS: 8-10 illustrate the inclusion the SEQ ID NO:3 with additional flanking sequences.

The decoy sequences may be made from standard nucleotides as illustrated by SEQ ID NO:3. Alternatively, non-standard or modified nucleotides may also be employed. For example, in order to promote stability of the decoy, locked nucleotides may be employed in the synthesis of part or all of the decoy. Alternatively, nucleotide modifications such as mixed bone, LNA modified, phosphothioate, etc. may also be used in the construction of the decoys. Those of skill in the art will recognize that any and all such nucleotide variants may be used to make the chimeric decoys of the present invention, and the phrase “decoy ODN” or “decoy” is intended to include decoys containing all such modifications.

The decoy ODNs of the present invention may be used to inhibit the expression (or over-expression) of MMPs in cells both in the case where expression/over-expression is occurring, and in the case where expression/over-expression is likely to occur. For example, individuals suffering from a cancer in which the cancer cells over-express an MMP may be treated with decoy DNA in order to inhibit MMP expression in the cancer cells. Alternatively, individuals that are identified as having a predisposition to develop a disease associated with MMP expression (e.g. cancer) may be treated prophylactically in order to prevent, slow or decrease MMP expression. Cells that are treated with the decoys of the present invention may be in vitro or in vivo.

The invention further encompasses methods for inhibiting expression of a targeted MMP (which can be any MMP of interest) in cells, particularly in cells that over-express at least one MMP. The method includes the step of administering to a cell an amount of a decoy ODN sufficient to inhibit expression of the MMP. The inhibition may be complete (i.e. expression of the MMP may be completely arrested) or partial (i.e. the level of expression of the MMP may be reduced compared to the level of expression prior to administration of the decoy). In particular, the cell may be over-expressing the MMP (i.e. producing MMP at a level that is higher than comparable control or normal cells) prior to administration of the decoy ODN. Administration of the decoy reduces, decreases or lowers the level of expression of the targeted MMP compared to the level of expression of MMP prior to administration of the decoy, or in comparison to the level of expression in other comparable control or untreated cells. Those of skill in the art are well acquainted with the concept of comparing treated populations of cells with comparable untreated or control cells to determine whether or not a treatment has been effective. The level of expression of an MMP in a cell that previously over-expressed the MMP will be reduced either partially or totally, and in general, will be reduced by at least about 40% and preferably by at least about 100% compared to the level of expression prior to treatment of the cell, or in a control cell.

In preferred embodiments of the invention, the cells that are treated by the methods of the present invention are those in which an unwanted or harmful condition has developed or is likely to develop due to expression/over-expression of an MMP. The cells may be in vitro or in vivo, for example in a patient in need, of such treatment. Examples of such diseases or conditions include but are not limited to various types of cancer (e.g. metastatic melanoma, renal cell carcinoma, colorectal cancer, lung cancer, endometrial and ovarian cancers, and other metastatic tumors; as well as arthritic disorders, periodontal diseases, neurodegenerative diseases, etc. In a preferred embodiment of the invention, the cells are glioma cells that over-express an MMP such as MMP-1, and the disease is cancer, in particular brain cancer such as GBM. However, those of skill in the art will recognize that many undesirable conditions and diseases associated with expression/over-expression of MMPs may be ameliorated by the practice of the present invention, and the treatment of all such conditions and diseases is intended to be encompassed by the present invention. Further, treatment of cells by the methods of the present invention may be carried out by itself, or in conjunction with other treatment modalities. For example, when treating cancer, the cancer or tumor cells may also be treated by other means such as chemotherapy, radiation, various surgical procedures, etc. Also, more than one type of decoy (i.e. decoys with varying sequences) may be administered at one time.

Administration of the decoy ODNs of the present invention may be carried out by any of many means that are known to the skilled practitioner. For example, delivery of the chimeric decoy may be carried out by direct infusion into the tumor, directed targeting using lipid carriers, etc.

Those of skill in the art will recognize that the decoy ODNs of the present invention may be administered in a composition that is suitable for the method of administration that is being used. Compositions containing the decoy ODNs for administration may also contain various other elements or compounds that facilitate administration or that help to preserve the composition. Examples include but are not limited to preservatives such as ascorbic acid, buffering agents, colorants, various charged species (e.g. salts, amino acids, etc.) that maintain a suitable ionic strength, modifications to the ODN that increase stability, and the like. The decoy ODN will be present in such a composition in an amount ranging from about 1-99%.

Those of skill in the art will recognize that the amount of decoy ODN to be administered will vary depending on the particular circumstances of the treatment. For example, when administered to a patient in vivo, the amount will vary depending on such factors as the patient's age, gender, general health, the disease being treated, the stage of the disease, the location of the cells being treated, and the like.

### EXAMPLES

### Example 1

Purpose: Inhibition of MMP-1 transcriptional activity in human glioma cell lines by novel SNP transcription factor decoys.

Methods: Decoys were designed to mimic position −1622 to −1603 of the MMP-1 promoter. The transcription factor decoy contains both ETS and AP1 DNA consensus sites as well as MMP-1 specific flanking sequences. Oligonucleotide probes for transcription factors, Ets-1, AP1, and MMP-1 promoter probes were obtained. Electromobility shift assays (EMSAs) were used to identify binding characteristics of the decoy. In addition, the effect of decoy treatment on transcriptional activity of MMP-1 was tested using a Dual Luciferase Reporter Assay. The two MMP-1 promoter constructs consist of pGL3 vectors with a −4,372 bp fragment of the MMP-1 promoter containing either the 1G sequence or the 2G sequence. Glioma cells were transfected using Lipofectamine 2000 with the MMP-1 promoter constructs and cotransfected with Renilla gene (pRL-TK) as an internal control. After addition of promoter the cells were treated with 5 uM decoy, and relative luciferase units (RLU) were recorded. The effect of decoy on MMP-1 protein expression was tested using a sandwich enzyme-linked immunoassay (ELISAs) on two glioma cell lines U87 (2G/2G) and T98 (1G/1G). Statistical analysis was done for both promoter and protein results using a 2-way ANOVA to conclude the effect of decoy on MMP-1 activity and expression.

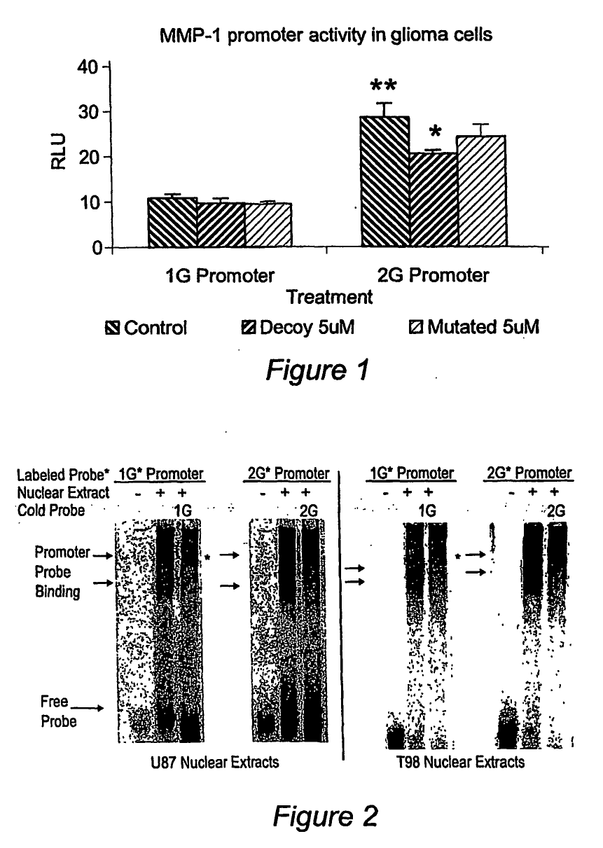

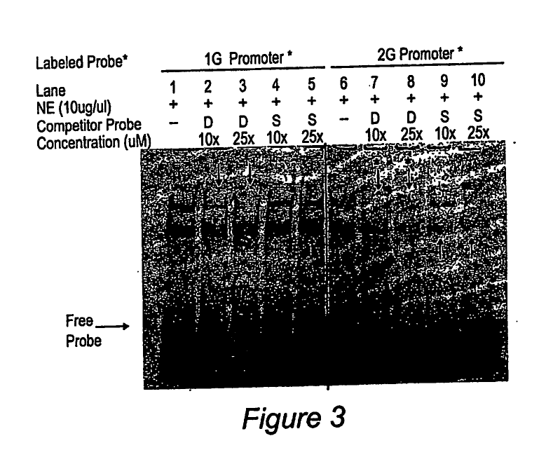

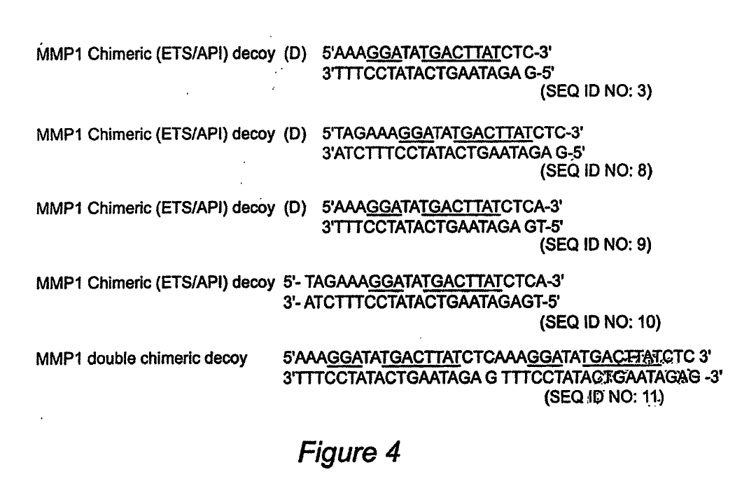

Results: EMSA results indicate that the transcription factor probes for Ets-1 and AP1 effectively bind proteins from glioma cell nuclear extracts. MMP-1 promoter probes bind to glioma cell nuclear proteins. In competitive EMSA experiments, excess decoys were able to inhibit nuclear protein interactions with the 2G MMP-1 promoter probe and not the 1G promoter probe, whereas the scramble decoy had no effect. Similar results are seen in U87 nuclear extracts. Promoter studies done in glioma cells revealed a significant increase in transcriptional activity after transfection with the 2G promoter, but not the 1G promoter (FIG. 1).

### Example 2

### Introduction

In recent years, despite the many advances in therapeutic and surgical techniques for glioblastoma multiforme (GBM), this form of brain cancer still remains incurable. Surgical resection, radiation and chemotherapy, still remain the standard treatment modalities for these patients; yet these therapies can have many disadvantages. Although these treatments have improved patients quality of life, these cancers still have the highest mortality rate, and one of the lowest survival rates.¹ A hallmark feature of GBM that underlies their malignant behavior is the ability of the glioma cells to infiltrate surrounding brain tissue.² The invasive nature of glioma cells is a key challenge in considering treatment for patients with GBMs. Because of the highly invasive nature and metastatic ability of the brain cancer, it would be beneficial to identify novel therapeutic approaches for glioblastomas.

Research has shown that glioma cells express a wide variety of proteinases including members of the matrix metalloproteinases (MMPs) family.³⁻⁶ Matrix metalloproteinases have been shown to be upregulated in many pathological conditions; such as periodontitis, atherosclerosis, arthritis and cancer. The increased levels of MMPs found in cancer usually results in aberrant connective tissue destruction, therefore indicating MMPs involvement in tumor invasion and metastasis.³ In particular, the gene encoding MMP-1 contains a functional SNP at −1607 and has been correlated with highly metastatic melanoma^{7,8}, renal cell carcinoma⁹, invasiveness of colorectal cancers, enhancement of lung cancer susceptibility¹¹, and also has been shown to be of prognostic importance in endometrial¹² and ovarian cancers.¹³ This functional SNP at −1607 (“2G” allele) creates a Ets DNA consensus-binding site, which has been shown to result in significantly higher levels of MMP-1 expression.¹⁴⁻¹⁶ An adjacent AP1 site at −1602 has been shown to cooperate with the Ets site on the MMP-1 promoter, which further aids in transcriptional regulation of MMP-1.¹³ As shown in Example 1, there is a significant increase in the 2G MMP-1 genotype in GBMs when compared to normal distributions In addition, in glioma cell lines the 2G SNP correlates with an increase in MMP-1 transcriptional activity. Therefore targeting transcription factors involved in the regulation of MMP-1 could be a beneficial method for the treatment of GBMs.

Transcription factor decoys (TFDs) have been used as treatment applications to target particular transcription factors involved in a variety of diseases. The decoys target nuclear proteins that are involved in the regulation of gene expression by having a positive or negative act on the promoter site of the target gene. One of the problems associated with decoys is the non-specific effects associated with the multiple number of genes regulated by specific transcription factors, caused due to either multiple number of transcription factors that regulate any gene or vice versa. Therefore, specificity towards target gene becomes an even greater a challenge. In addition, targeting a particular tissue type is also problematic. The problem enhanced when systemically delivering the drug because most genes are expressed in a wide variety of tissues. A number of studies have been reported in the literature emphasizing the use of transcription factor decoys as potential therapeutics for the treatment of disease. The most promising decoy drug to date is the E2F decoy. The TF E2F plays an important role in the regulation of cell cycle progression and transition from G1 to S-phase. Investigators have shown that the E2F decoy was able to inhibit neointimal hyperplasia, a common factor in vein graft failures.¹⁸ Treatment with E2F decoy later showed preservation in vein graft endothelial function, and a prevention of diet-induced atherosclerosis in rat models.¹⁸ A randomized, placebo control phase II clinical trial using E2F decoy showed a significant reduction in long term morbidity and mortality associated with human coronary artery vein graft failure.¹⁹ In addition the decoy showed no adverse events, this due to its applied local delivery to target tissue.¹⁹ Other decoy studies against activating protein-1 (AP-1), cAMP-response element-binding protein (CREB), STAT proteins, erythroblastosis twenty-six (ETS) and nuclear factor κB (NFκB) have also proven effective in the treatment of a variety of pathogenesis's. Use of these proteins as regulatory targets have shown effects such as inhibition of CREB proteins to suppress tumor cell activity²⁰, and inhibition of ETS proteins in hypoxia induced transcription of genes to inhibit carcinogenic and angiogenic properties.²¹ The most targeted regulatory factor, using a decoy approach, has been the NFκB transcription factors. Investigations have shown that a NFκB decoy was able to block expression of TGF-alpha, IL-2, and IL-6 (common cytokines in inflammatory responses) suggesting the use of the decoy to treat autoimmune diseases.²² Additionally, NFκB decoys have been used to treat glioblastomas. A successful approach was that in which a biodegradable polymer microparticle was used to deliver the decoy to the brain and inhibit NFkB activity.²³ The NFkB decoy was able to disregulate GBM cell growth by decreasing cyclin D and cdk expression (genes promoting progression thru the cell cycle), and inhibit effects of GBM cell growth.²³ Although this approach seemed advantages the investigators neglected investigate non-specific effects caused by the decoy, a crucial aspect since NFκB regulates many genes.

The use of decoys as therapeutic interventions have proven effective, yet the predominant challenge of eliminating non-specific effect still remains. Non-specific effects and more importantly specificity toward target gene becomes the ultimate test when designing decoy drugs. The E2F decoy is the most promising because it overcomes both challenges. The investigators used a pressure mediated delivery in an ex-vivo model, treatment of the vein outside of the body. A method that results in limited (if any) amounts of the decoy entering systemic circulation, thereby a targeted delivery with no few adverse effects. The NFkB decoy for the treatment of GBMs also proved to be a good local delivery system, and in addition a protective agent against nuclease degradation, using the biodegradable polymer microparticle, although, there was no indication that non-specific effects the decoy had on other genes was investigated.

In addition to targeting glioma tumors, the present novel SNP decoy/drug has the potential to target MMP-1 increased expression and possibly tumorigenicity found in cancers such as lung, endometrial, ovarian, breast, renal, colon, and skin. Studies showing that ETS and AP1 transcription factors at −1607 and −1602, work in together on the regulation of MMP-1 expression provide evidence of the potential that this decoy will work.¹³ Due to the sequence homogeneity of the decoy to the MMP-1 promoter we hypothesized that in the presence of an excess amount of decoy in glioma cells, it would be possible to “trick” the ETS and AP1 protein complex into preferably binding to decoy instead of the promoter. In the present study, experiments were designed to determine whether our specific ETS/AP1 transcription factor decoy would serve as bait for ETS-1 and AP1 transcription factors that would normally target the MMP-1 promoter, thereby interfering with transcriptional activity. A phosphorothioate (PS) modified double stranded transcription factor decoy oligonucleotide (ODN) that matches the functional SNP at −1607 of the MMP-1 promoter in addition to the adjacent AP1 site was used to investigate the following hypothesis: The increase in MMP-1 transcriptional activity that results from the polymorphism at −1607 can be inhibited by the use of a specific transcription factor decoy. Experiments were designed to test “proof of principle” in vitro effects.

### Materials and Methods

### Oligonucleotides and Decoy Design

Transcription factor and promoter probes: All probes were confirmed for sequence specificity using nucleotide BLAST® sequencing by NCBI. All probes were synthesized commercially, annealed and biotinylated (Biosynthesis) before shipment. Table 1 provides nucleotide sequence for each of the probes.

Decoy design: The 2G promoter site sequence containing the ETS consensus binding site were analyzed using nucleotide BLAST® to verify sequence specificity to MMP-1 gene. Control decoys scrambled and mutated sequences were analyzed to confirm they did not identify with MMP-1. All decoys were synthesized commercially (Biosynthesis), with three nucleotides on both 3′ and 5′ ends containing Phosphothioate (PS) modification to aid in stability.

Cell culture and Nuclear Extracts

The following cell lines are commercially available; currently used in our lab and for the present study: The human glioblastoma: cell lines U87-MG and T98 were obtained from ATCC. All cell lines were maintained in culture with Dulbeco's Modified Eagles Medium (DMEM) supplemented with 10% fetal bovine serum (FBS), gentamyacin (50 mg/ml) and non-essential amino acids (L-glutamine) (200 mM) (Gibco).

Nuclear extracts were prepared from each of the cell lines mentioned above. After treatment cells were thoroughly washed in phosphate buffer saline (PBS) and nuclear extracts isolated following standard procedure in hypotonic buffer (Sigma Aldrich). The concentration of protein in nuclear extracts was determined by DC protein assay (BioRad).

### Electromobility Shift Assays (EMSA)

Binding assays were preformed using a standard electromobility shift assay (EMSA) protocol. In brief, nuclear extract proteins (2-10 ug) were preincubated with binding buffer and non-specific inhibitors (poly dI-dC). Binding activity was assessed by incubating the reaction mixture with biotin labeled probes (10 ng/uL) for 20 minutes at 20° C. The protein-DNA complexes were separated on a 6% DNA retardation gel (Invitrogen). Complexes were then transferred onto nylon membranes and cross-linked using (UV-crosslinker). Membranes were blocked using Odyssey Blocking Bluffer (1:1 with PBS), then incubated with Steptavidin antibody complexed with 800 dye, then washed in 0.5% PBS-Tween and read directly. Detection of bands was done using Odyssey imaging system at 800 nm (Licor Corp.). Control binding was assessed by addition of excess unlabelled probes (300 ng/ul) to reaction mixture, to compete out the binding of labeled probe to NE protein. Transcription factor probe sequences and promoter probe sequences are shown in Table 1.

### Table 1: MMP-1 Promoter Design and Decoy Design

Highlighted regions identified as Ets and Ap1 family binding sites, respectively, within Both the MMP promoter probes and the specific ETS/AP1 decoy. Bold/italics regions identify mutated oligonucleotides within binding region. Three bases at each end 5′ and 3′ of the decoy sequences have been modified with phosphorothioated (PS) to aid in stability of molecules when treated with cells (Bold). Decoys are represented by D=ETS/AP1 decoy, M=ETS and AP1 mutated Decoy, and S=Scrambled Decoy.

### Promoter Probe Oligonucleotides:

2G MMP-1 Promoter Probe

(SEQ ID NO: 1)

5′- TAGAAAGGATATGACTTATCTCA-3′
3′- ATCTTTCCTATACTGAATAGAGT-5′
1G MMP-1 Promoter Probe

(SEQ ID NO: 2)

5′- TAGAAAGATATGACTTATCTCA-3′
3′- ATCTTTCTATACTGAATAGAGT-5′

### Decoy Oligonucleotides:

(ETS/API) decoy (D)

(SEQ ID NO: 3)

5′AAAGGATATGACTTATCTC-3′
3′TTTCCTATACTGAATAGA G-5′
Scrambled decoy (S)

(SEQ ID NO: 4)

5′- CTGCGTAGCGAATTAATATGAC-3′
3′- GACGCATCGCTTAATTATACTG-5′
Mutated decoy (M)

(SEQ ID NO: 5)

[symbol]

3′-TTT C TTATAGAGACTAGAG-5′

### Transcription Factor Oligonucleotides:

ETS Transcription Factor

(SEQ ID NO: 6)

5′GGAGGAGGGCTGCTGCTTGAGGAAGTATAAGAAT-3′
3′-CCTCCTCCCGACGACGAACTCCTTCATATTCTTA-5′
AP1 Transcription Factor

(SEQ ID NO: 7)

5′-CGCTTGATGACTCAGCCGGAA-3′
3′-GCGAACTACTGAGTCGGCCTT-5′

Competitive EMSA Assays to Determine Decoys Ability to Interfere with Binding

EMSA assays were done as described above, except that labeled probes were used as the 1G and 2G promoter probes and an excess of decoys were added as competitor probes. The decoys used in these experiments were the D-ETS/AP-1 and S-Scrambled decoys (Table 1). Binding activity was assessed by incubating the reaction mixture with the biotin labeled promoter probes (10 ng/ul), competition lanes had an addition of decoys at 100 ng/ul (10×), and 250 ng/ul (25×). Detection of bands was done using the Odyssey imaging system at 800 nm. Determination of relative band intensities was done using Odyssey software (Licor Corp.).

### Transcriptional Activity/Promoter Study Assays

MMP-1 promoter activity is examined by luciferase promoter/reporter assays. Transfection of Cells and Luciferase Assays for Transcriptional Activity: U87 cells, (2×10⁵ cells/24 well plate) are plated. The two MMP-1 promoter constructs consist of pGL3 vectors with a 4,372 bp fragment of the MMP-1 promoter containing either the 1G sequence or the 2G sequence. U87 cells are transfected using Lipofectamine 2000 with the MMP-1 promoter constructs and as an internal control the Renilla gene is co-transfected (pRL-TK) with each of the MMP-1 promoter constructs. In addition, the decoy (D, M, and S) drugs are cotransfected with the promoter constructs. Twelve and twenty-four hours after addition of the TF decoys, cells are lysed and subjected to the Dual-Luciferase Reporter Assay System (Promega). Detection of transcriptional activity is shown by comparing relative luciferase units of each transfected cell line with and without treatment as a ratio of luciferase activity to Renilla control vector activity.

### Protein Expression—ELISA Assays

U87 and T98 cells were plated on 24 well plates in 10% no phenol, no antibiotics DMEM, after four hours cells were then placed in 5% media overnight. The next morning media was removed and replaced with fresh 2% media, and treated with various concentrations of decoys; twelve hours later an additional dose of decoy was added to each well. Twenty-four hours after initial treatment, conditioned media was collected and analyzed for MMP-1 protein expression using a sandwich enzyme-linked immunoassay (ELISA). In brief, 200 ng/well of capture monoclonal MMP-1 antibody (Accurate Scientific cat# ACLMABMMP1) were added to each well of a 96 well plate and placed in 4 C overnight. Next morning plates were blocked by adding 250 uL of PBS with 0.5% BSA for 2 hours at room temperature. Plates were aspirated and washed, and 100 uL of standard (recombinant MMP-1 protein, R&D systems cat# 901-MP-010) and samples were added to each well. Standards and samples were incubated for one hour at 37 C, plated and washed, and detection antibody was added. 100 uL of 1 ug/uL biotinylated detection antibody (Accurate Scientific cat# ACL2MMP1) was added to each well and samples were incubated at room temperature for one and a half hours. Plates were then washed, and 100 uL diluted 1:1000 streptavidin HRP (Sigma) was added to each well, followed by incubation for 30 minutes at room temperature. Plates were again washed, and 100 uL of chromagen (TMB-Sigma) was added to each well and to induce color change. Upon visible color change, the reaction was stopped with 2.5N H₂SO₄. Plates were read at 450 nm within thirty minutes of stopping the reaction.

### Statistical Analysis

All numerical values reported represent mean and standard deviation (SD). Statistical analysis was done for both promoter and protein results using a 2-way ANOVA to conclude the effect of decoy on MMP-1 activity and expression. Asterisks were used to graphically denote statistical significance (p<0.05) on figures.

### Results

Transcription Factor Probes for Ets-1, AP1 Effectively Bind Proteins from Glioma Cell Nuclear Extracts.

In the first set of experiments we examined the binding of nuclear extracts to DNA probes for ETS and AP1, and as control NFkB, transcription factors. Electromobility assays revealed that nuclear extracts isolated from U87 and T98 glioma cells bind to the probes, resulting in a band shift of the biotin labeled probe. The addition of cold unlabeled DNA probes resulted in competition of the band shift result, demonstrating specificity to the probes. Results verify that the transcription factors of interest, ETS-1 and AP-1, are present within the two glioblastoma cell lines. In addition, EMSAs reveal that MMP-1 promoter probes containing either the 1G or 2G sequence bind to glioma cell nuclear extracts from both T98 and U87, and these interactions are competed out with excess unlabeled probes.

In addition, the results indicate an increase in the relative band intensity of both the upper and lower bands (not shown) with addition of the 2G promoter probe when compared to the 1G promoter probe, in both the U87 and T98 gels. Additionally, the number of retarded bands increases in the U87 and T98 nuclear extracts binding to the 2G promoter probe (results not shown). These results further imply the potential of the 2G promoter to have an increase in transcriptional activity. The EMSA results suggest that the lower retarded band represents the occupation of one protein bound (lower MW) to a probe, and the upper retarded band is indicative of both proteins being bound (higher MW). The EMSAs reveal less activity of the upper band in the 1G as compared to the 2G. This result suggests that with the addition of the 2G probe both the ETS and AP1 nuclear protein are able to bind cooperatively and aid in the increase in MMP-1 transcription.

Competitive EMSA Experiments in which Labeled 2G MMP-1 Promoter Probes in the Presence of Excess Transcription Factor Decoy were Able to Inhibit Interactions of the 2G MMP-1 Promoter with Proteins from Human Glioma Nuclear Extracts.

Competitive EMSA assays have been used to show specificity of decoy oligonucleotides to 2G MMP-1 promoter probes compared to 1G. Using the 1G promoter as the labeled probe competitive EMSAs revealed the ETS/AP1 decoy and scrambled decoy had limited effect in competing out binding to the 1G promoter probe. The ETS/AP1 decoy began to compete out the 1G band shift result at very high doses. In contrast, using the 2G promoter as the labeled probe competitive EMSAs reveal the ETS/AP1 decoy was effectively able to compete out binding to the 2G probe. The scrambled decoy was unable to inhibit the interactions to the 2G promoter probe. Results of the specificity of the decoy to the 2G promoter probe was seen using both T98 and U87 nuclear extracts. In addition, relative band intensity of the upper band of both 1G and 2G promoter probe further identified the decrease in binding activity seen when band shift is competed out with ETS/AP1 decoy.

The 2G SNP Correlates with Increases in MMP-1 Transcriptional Activity and Increase Transcription can be Inhibited by ETS/AP1 Decoy in Glioma Cells.

Promoter studies have been conducted using MMP-1 promoter/reporter constructs. In the two glioma cell lines tested (U87 and T98), there was a significant increase in luciferase reporter activity in both U87 and T98 cells transfected with the 2G MMP-1 promoter construct compared to the 1G promoter construct (Table 2). When promoters were transfected into T98 cells the 2G promoter activity increased when compared to 1G promoter activity; mean relative luciferase units (RLUs) were 21.34 and 31.76, respectively. In addition, T98 cells cotransfected with the 1G and 2G promoter constructs and decoys there was a significant decrease in both 1G and 2G promoter probe activity (Table 2). When T98 cells were cotransfected with the mutated decoy, a significant decrease in activity was seen with the 1G promoter 21.34 to 8.69 (p<0.0001), but not the 2G 31.76 to 20.74 (p=0.2167).

Similar results were seen when promoters were transfected into U87 cells. A significant increase in the 2G promoter activity compared to the 1G was seen with almost a two fold increase in transcription, 135.85 and 84.7, respectively. Additionally in U87 cells cotransfected with the promoter constructs and the decoys, there was a significant decrease in both the 1G and 2G activity when treated with the decoy. Although a more substantial effect was seen in the 2G, 1G promoter activity decreased by 15.00 RLU (an 18% decrease), and the 2G promoter activity decreased by 47.36 (a 35% decrease). When U87 cells were cotransfected with the mutated decoy, a significant effect on 1G promoter activity but not 2G promoter activity was again observed.

TABLE 2
Summary of promoter results in glioma cell lines. Statistical analysis
was done using 2-way anova to account for variability within each
experiment. Means and 95% CI reported. There was a significant increase
in transcription of cells with the addition of the 2G promoter probe
when compared to the 1G (indicated by * p < 0.0001). Addition
of the decoy was able to significantly decrease in transcription
of the1G promoter, and also the 2G promoter in both of the cell lines
tested (p-values indicated on chart).

| | | | | |
|---|---|---|---|---|
| Treatment Value | Mean | SE | 95% CI | p value |

T98 Cell Line Transcriptional Activity
1G Promoter

| | | | | |
|---|---|---|---|---|
| Control | 21.34 | 3.39 | (14.07, 28.62) | |
| Decoy 5 μM | 9.35 | 0.84 | (7.73, 11.34) | >0.0001 |
| Mutated 5 μM | 8.69 | 1.36 | (7.06, 12.06) | >0.001 |

2G Promoter

| | | | | |
|---|---|---|---|---|
| Control | 31.76 | 4.08 | (22.99, 40.56) | * >0.0001 |
| Decoy 5 μM | 16.45 | 1.82 | 12.53, 20.37) | 0.0001 |
| Mutated 5 μM | 20.74 | 1.26 | (17.95, 23.56) | 0.2167 |

U87 Cell Line Transcriptional Activity
1G Promoter

| | | | | |
|---|---|---|---|---|
| Control | 84.7 | 12.31 | (66.27, 103.13) | |
| Decoy 5 μM | 69.69 | 8.28 | (51.27, 86.36) | 0.0082 |
| Mutated 5 μM | 63.79 | 7.17 | (41.30, 86.36) | 0.065 |

2G Promoter

| | | | | |
|---|---|---|---|---|
| Control | 135.85 | 9.58 | (117.43, 154.28) | >0.0001 |
| Decoy 5 μM | 88.49 | 9.28 | (70.06, 106.92) | 0.0001 |
| Mutated c | 95.64 | 11.74 | (73.07, 118.21) | 0.18 |

* 1G vs 2G

### Decoys Ability to Inhibit MMP-1 Protein Expression in Glioma Cell Lines

Studies were carried out to ascertain whether the decoys had the ability to decrease MMP-1 protein expression in two cell lines. The results from ELISA experiments further indicate the ability of the SNP decoy to decrease the MMP-protein as shown in Table 3, the SNP Decoy having the most effect on the expression of MMP-1 protein compared to the two control decoys, mutated and scrambled. The treatment of both cell lines with doses of 5 uM and 10 uM of the novel SNP decoy was effectively able to decrease MMP-1 expression in a dose dependent manner, 10.29% and 15.33% in T98, and 30.5% and 46.25% in U87, respectively. Additionally, the novel SNP decoy was three times more effective at both doses in inhibiting MMP-1 protein expression in the 2G/2G cell line, U87, when compared to the 1G/1G cell line, T98. At higher doses of 10 M the scrambled control decoys were also able to significantly inhibit MMP-1 protein expression in T98 cells (20.03% decrease), but had limited effect in U87 cells (12.67% decrease).

TABLE 3
Knockdown of MMP-1 protein expression in T98 and U87 cell lines.
Summary of ELISA assays of MMP-1 Protein expression in U87 and T98
cells treated with decoys. Double asterisks-indicates significant
difference when compared to control p < 0.05. Higher doses control
decoys were able to significantly decrease protein expression.
MMP-1 Protein Expression

| | | T98 Cells 1G/1G | | U87 Cells 2G/2G | |
|---|---|---|---|---|---|
| | | % Protein Decrease | | % Protein Decrease | |
| | | Relative to Control | | Relative to Control | |
| Treatment | N | | SD | | SD |
| Decoy (5 μM) | 4 | 10.29 | 12.22 | 30.5** | 11.47 |
| Decoy (10 μM) | 4 | 15.33** | 11.87 | 46.25** | 8.7 |
| Mutated Decoy (5 μM) | 2 | 1 | | 19 | |
| Mutated Decoy (10 μM) | 2 | 1 | | 23.5 | |
| Scrambled Decoy (5 μM) | 3 | 9 | 6 | 6.4 | 33.71 |
| Scrambled Decoy | 3 | 20.03** | 15.1 | 12.67 | 29 |
| (10 μM) | | | | | |

### Discussion

This study was designed to address the hypothesis that the increase in transcriptional activity due to the 2G functional SNP can be inhibited using specific ETS/AP1 decoys. The study was undertaken to answer the following questions; 1.) Can specifically designed decoys selectively inhibit nuclear protein binding to the 2G promoter and not the 1G promoter? 2.) Do decoys affect the transcriptional activity of the MMP-1 promoter? and 3.) Do ETS/AP1 decoys effectively inhibit MMP-1 protein expression in GBM cells, and is inhibition specific to the 2G genotype?

Researchers have reported that the SNP present at position −1607, creates within the MMP-1 promoter an ETS DNA binding site which results in significantly higher levels of MMP-1 expression, due to the increase in transcriptional activity.⁵ The increase in MMP-1 expression and possibly tumorigenicity due to the functional SNP within the promoter site has been extensively studied in cancers such as lung, endometrial, ovarian, breast, renal, colon, skin, but little is known about this SNP in the brain. Results from our laboratory indicate that a significant number of GBM tissues contain a 2G allele when compared to control samples.⁴ In addition, an adjacent AP1 site at −1602 has been shown to cooperate with the ETS site, which further aids in transcriptional regulation of MMP-1.³ The studies described here demonstrate that both ETS-1 and AP1 transcription factors are present within two Glioma cell lines. Whether or not the nuclear proteins present within the two GBM cell lines could bind to the promoters with and without the SNP was also studied. The results showed an increase in activity on the 2G promoter when compared to the 1G promoter, indicating the ability of both TF proteins to bind to the promoter and up-regulate transcriptional activity. This result is consistent with other reports in which the addition of PMA treated amnion mesenchymal cells was able to increase the levels of protein with high affinity to the 2G probe.²⁴ That same study also indicated that c-jun (a unit of the AP-1 protein) was able to enhance the binding of recombinant Ets-1 protein to the 2G promoter, further indicating a cooperative interaction between both the Ets and AP1 site on the promoter. The fact that both the Ets-1 and AP1 TFs are present within the cell lines utilized in the present investigations, and the increase in the 2G promoter binding, further suggests this cooperation.

MMP-1 can be regulated at many levels; such as transcriptional, post transcriptional, translational, secretion, activation, and degradation/metabolism. In normal cells activation and expression of MMP-1 is predominantly influenced by growth factors such as; bFGF, HGF, EGF, IL-1, and TNFα.²⁶ The induction of this pathway requires the activation of the mitogen-activated protein kinase (MAPK) pathway, that eventually activates many of the ETS and AP1 transcription factors involved in MMP-1 expression.^{7,27,29} The MAPK pathway consists of three major families: the Extracellular response kinase (ERK), p38, and the Jun. N-terminal kinase (JNK).³¹ Two of the three pathways are primarily involved in regulation of MMP-1; the JNK pathway which can activate AP1 transcription factors, and the ERK pathway which activates many of the ETS family of transcription factors. Many transcription factors are involved in MMP-1 expression, some playing indirect role in the regulation. For example, p53 has been shown to inhibit c-fos expression³² while elk1 and elk2 can bind to the c-fos promoter and enhance its transcription.³³ Additionally, it is not only ETS and AP1 that directly bind the promoter and enhance transcription; rather, other transcription factors such as NFκB and CREB can also bind the MMP-1 promoter and effect transcription of MMP-1.^{34,35} Although a lot has been elucidated concerning normal cellular expression of MMP-1, much less is understood about regulatory mechanisms involved in the enhanced expression of MMP-1 in cancerous cells. The present work focuses on ETS and AP1 transcription factor binding sites within the promoter. However, other of the identified sites may also be useful targets for the practice of the present invention.

The present studies demonstrate that the treatment of glioma cells with novel SNP decoys at 5 μM concentration can effectively decrease the MMP-1 2G promoter activity up to 50% in T98 and 35% in U87 cells.

In addition, these studies also demonstrate the ability of the decoys to inhibit MMP-1 protein expression in glioma cell lines containing the 2G allele. The novel SNP decoy designed to bind ETS-1 and AP1 resulted in a significantly lower MMP-1 protein expression compared to control decoys. The treatment of both cell lines with varying doses of novel SNP decoy was effectively able to decrease MMP-1 expression in a dose dependent manner. In addition, the novel SNP decoy was three times more effective in inhibiting MMP-1 protein expression in the 2G/2G cell line, U87, when compared to the 1G/1G cell line, T98. These results support the genotype specificity of the novel SNP decoy.

This example demonstrates that transcription factor decoys designed to be specific for MMP-1 SNP and flanking sequences can inhibit nuclear extract protein/MMP-1 promoter DNA binding, MMP-1 (2G) transcriptional activity and MMP-1 protein expression in glioma cells containing a 2G allele. The findings presented here warrant the use of this novel SNP decoy as a genotype specific drug for the inhibition of MMP-1 protein in brain and other cancers.

### REFERENCES FOR EXAMPLE 2

1 Pollack I F, Biegel J, Yates A, Hamilton R, Finkelstein S: Risk assignment in childhood brain tumors: the emerging role of molecular and biological classification. Current Oncology Report 2002, 4(2): 114-122.
2 Visted, T., Enger, P. O., Lund-Johansen, M., Bjerkvig, 3: Mechanisms of tumor cell invasion and angiogenesis in the central nervous system. Front Biosci 2003, 1(8): e289-304.
3 Coussens L M, Fingleton B, Matrisian L M: Matrix metalloproteinase inhibitors and cancer: Trials and tribulations. Science 2002, 295(29): 2387-2392.
4 Liotta L A, Tryggvason K, Garbrisa S, Hart I, Foltz C M, Shafie S: Metastatic potential correlates with enzymatic degradation of basement membrane collagen. Nature 1980, 284:67-68.
5 Curran S, Murray G I: Matrix metalloproteinases in tumor invasion and metastis. J. of pathology 1999, 189:300-308.
6 Stetler-Stevenson W G: The role of matrix metalloproteinases in tumor invasion, metastasis, and angiogenesis. Surgical Oncology Clin. N. Am 2001, 10(2):383-392.
7 Rutter J L, Mitchell T I, Buttice G, Meyers J, Grusella J F, Ozelius L J, Brinckerhoff C E: A single nucleotide polymorphism in the matrix metalloproteinase-1 promoter creates and Ets binding site and augments transcription. Cancer Research 1998, 58:5321-5325.
8 Ye S, Dhillon S, Turner S J, Bateman A C, Theaker J M, Pinckering R M, Day I, Howell W M: Invasiveness of malignant melanoma is influenced by matrix metalloproteinase-1 gene polymorphism. Cancer Research 2001, 61:1296-1298.
9 Hirate, H., Naito, K., Yoshihiro, S., Matsuyama H., Suchiro, Y., and Hinoda, Y. A single nucleotide polymorphism in the matrix metalloproteinase-1 promoter is associated with conventional renal cell carcinoma. Int. Journal of Cancer 2003, 106(3):372-374.
10 Zhu Y, Spitz M R, Lei L, Mills G B, Wu X: A single nucleotide polymorphism in the matrixmetalloproteinase-1 promoter enhances lung susceptibility. Cancer Research 2001, 61:7825-7829.
11 Zhu Y, Spitz M R, Lei L, Mills G B, Wu X: A single nucleotide polymorphism in the matrixmetalloproteinase-1 promoter enhances lung susceptibility. Cancer Research 2001, 61:7825-7829.
12 Kanamori Y, Matshushima M, Minaguchi T, Kobayasm K, Sagae S, Kudo R, Terakawa N, Nekamura Y: Correlation of matrix metalloproteinase-1 gene in ovarian cancer and an insertion deletion polymorphism in its promoter region. Cancer Research 1998, 39:42254227.
13 Nismoka Y, Kobayashi K, Sagae S, Ishioka S, Nishikawa A, Matsushima M, Kanamori Y, Minaguchi T, Nekamura Y, Tokino T, Kudo R: A single nucleotide polymorphism in the matrix metalloproteinase-1 promoter in endometrial cancers. Japan J. of Cancer Research 2000, 91:612-615.
14 Behrens P, Wellman M R, Krischler J, Wernert N: The ets-1 transcription factor is up-regulated together with MMP 1 and MMP 9 in the stroma of pre-invasive breast cancer. J. of Pathology 2001, 194:43-50.
15 Iwasaka C, Tanaka K, Abe M, Sato Y: Ets-1 regulates angiogenesis by inducing the expression of urokinase-type plasminogen activator and matrix metalloproteinase-1 and the migration of vascular endothelial cells. J. of Cellular Physiology 1996, 169:522-531.
16 Watabe T, Yoshida K, Shindoh M, Kaya M, Fujikawa K, Sato H, Seiki M, Ishii S, Fujinaga K: The Ets-1 and Ets-2transcription factors activate the promoters for invasion-associated urokinase and collagenase genes in response to epidermal growth factor. Int. J. of Cancer 1998, 77:128-137.
17 Fillmore H, McCready J, Sykes V, Broaddus W: A single nucleotide polymorphism (SNP) in the MMP-1 promoter influences MMP-1 expression in human gliomas. Abstract Neuro-Oncology 2002, 4:328-329
18 Mann M J, Dzau V J: Therapeutic applications of transcription factor decoy oligonucleotides. The J. of Clinical Investigation 2000, 106:1071-1075.
19 Ehsan, A., M. J. Mann, G. Dell'Acqua, and V. J. Dzau. (2001). Long-term stabilization of vein graft wall architecture and prolonged resistance to experimental atherosclerosis after E2F decoy oligonucleotide gene therapy. Journal of Thoracic Cardiovascular Surgery, 121, 714-722.
20 Cho, Y. S., Kim, M. K., Cheadle, C., Neary, C., Park, Y. G., Becker, K. G., and Cho-Chung, Y. S. A genomic-scale view of the cAMP response element enhancer decoy: a tumor target-based genetic tool. Proc. Nat. Acad. Sci. 2002, 9(24): 15626-15631.
21 Oikawa M, Abe M, Kurosawa H, Hida W, Shirato K, and Sato Y. Hypoxia induses transcription factor ETS-1 via the activity of the hypoxia-inducible factor-1. Biochemical and Biophysical Research Communications 2001. 289: 3943.
22 Khaled A R, Butfiloski E J, Sobel E S, and Schiffenbauer, J. Use of phosphotioate-modified oligonucleotides to inhibit NFkB expression in lymphocyte function. Clin. Immunol. Immunopathol. 86:170-179.
23 Gill J S, Zhu X, Moore M, Lu L, Yaszemski M, Windebank A J. Effects of NFkB decoy aligonucleotides released from biodegradable polymer microparticles of a glioblastoma cell line. Biomaterials 2002. 23: 2773-2781.
24 Fugimoto T, Parry S, Urbanek M, Sammel M, Macones G, Kuivaneimi H, Romero R, Strauss J F 3rd. A single nucleotide polymorphism in the MMP-1 promoter influences amnion cell MMP-1 expression and risk for preterm premature rupture of the fetal membrane. J. Biol. Chem. 2002, 277 (8): 6296-6302.
25 Benbow, U., Rutter, J. L., Lowrey, C. H., and Brinckerhoff, C. E. Transcriptional repression of the human collagenase-1 (MMP-1) gene in MDA231 breast cancer cells by all-trans retenoic acid requires distal regions of the promoter. Brit. Journal of Cancer 1999, 79(2): 221-228.
26 Vencenti, M. P. White, L. A., Schroen, D. J. Benbow, U. Brinckerhoff C. E. Regulating expression of the gene for matrix metalloproteinase-1 (collagenase): mechanisms that control enzyme activity, transcription and mRNA stability. Crit. Rev Eukaryot Gene Expr 1996, 6: 391-411.
27 Aho, S., Rouda, S., Kennedy, S. H., Qin, H., Tan, E. M. Regulation of human interstitial collagenase (matrix metalloproteinase-1) promoter activity by fibroblast growth factor. Eur. J. Biochem. 1997. 247: 503-510
28 Benbow, U., Tower, G. B., Wyatt, C. A., Buttice, G., and Brinckerhoff, C. E. High levels of MMP-1 expression in the absence of the 2G single nucleotide polymorphism is mediated by p38 and ERK1/2 mitogen activated protein kinase in VMM5 melanoma cells J. Cell Biochem 2002 86, 307-19.
29 Brauchle, M., Gluck, D., Di Padova, F., Han, J., and Gram, H. Independent role of p38 and ERK1/2 mitogen activated protein kinases in the upregulation of matrix metalloproteinase-1. Exp Cell Res 2000, 258(1): 135-144.
30 Tower, G. B., Coon, C. C., Benbow, U. Vincenti M. P., and Brinckerhoff, C. E. ERK1/2 differentially regulates the expression from the 1G/2G single nucleotide polymorphism in the MMP-1 promoter in melanoma cells. Biochim Biophy Acta 2002, 1586: 265-274.
31 Johnson, G. L., and Lapadat R. Mitogen activated protein kinase pathways mediated by ERK, JNK, and p38. Science 2002, 298: 1911-1912.
32 Piu, F., Aronheim, A., Katz, S., Karin, M. AP-1 repressor protein JDP-2: inhibition of UV-mediated apoptosis through p53 down-regulation. Mol. Cell. Biol. 2001, 21(9):3012-24.
33 Galetic, I., Maira, S. M., Andjelkovic, M., Hemmings, B. A. Negative regulation of ERK and Elk by protein kinase B modulates c-fos transcription. J. Biol. Chem. 2003, 278(7): 4416-4423.
34 Vincenti, M. and Brinckerhoff, C. E. Transcriptional regulation of collagenase (MMP-1 and MMP-13) genes in arthritis: integration of complex signaling pathways for the recruitment of gene-specific transcription factors. Arthritis Research 2002, 4: 157-164.
35 Lai, W., Zhuo, M., Shankavaram, U., Peng, G., and Wahl, L. Differential regulation of lipopolysaccharide-induced monocyte matrix metalloproteinase (MMP)-1 and MMP-9 by p38 and Extracellular signal-related kinase 1/2 mitogen-activated protein kinase. J. of Immuno. 2003, 170:6244-6249.

While the invention has been described in terms of its preferred embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

1. A method of inhibiting expression of a Matrix Metalloproteinase (MMP) in a cell, comprising the step of
introducing into said cell an oligonucleotide, wherein said oligonucleotide binds at least two eukaryotic transcription factors, and wherein said at least two transcription factors bind to a promoter for a gene encoding said MMP.

2. The method of claim 1, wherein said MMP is MMP-1.

3. The method of claim 1, wherein said oligonucleotide binds Ap-1 transcription factor.

4. The method of claim 1, wherein said oligonucleotide binds Ets-1 transcription factor.

5. The method of claim 1, wherein said oligonucleotide binds both Ap-1 transcription factor and Ets-1 transcription factor.

6. The method of claim 1, wherein said oligonucleotide is SEQ ID NO:3.

7. The method of claim 1, wherein said oligonucleotide contains SEQ ID NO:3.

8. The method of claim 7, wherein said oligonucleotide is SEQ ID NO:8.

9. The method of claim 7, wherein said oligonucleotide is SEQ ID NO:9.

10. The method of claim 7, wherein said oligonucleotide is SEQ ID NO:10.

11. The method of claim 7, wherein said oligonucleotide is SEQ ID NO:11.

12. A chimeric decoy oligonucleotide, wherein said chimeric decoy oligonucleotide contains at least two binding sites for a eukaryotic transcription factor, and wherein said eukaryotic transcription factor binds to a promoter for a gene encoding an MMP.

13. The chimeric decoy ologonucleotide of claim 12, wherein said MMP is MMP-1.

14. The chimeric decoy ologonucleotide of claim 12, wherein said chimeric decoy oligonucleotide binds Ap-1 transcription factor.

15. The chimeric decoy ologonucleotide of claim 12, wherein said chimeric decoy oligonucleotide binds Ets-1 transcription factor.

16. The chimeric decoy ologonucleotide of claim 12, wherein said chimeric decoy oligonucleotide binds both Ap-1 transcription factor and Ets-1 transcription factor.

17. The chimeric decoy oligonucleotide of claim 12, wherein said chimeric decoy oligonucleotide is SEQ ID NO:3.

18. The chimeric decoy oligonucleotide of claim 12, wherein said chimeric decoy oligonucleotide contains SEQ ID NO:3.

19. The chimeric decoy oligonucleotide of claim 18, wherein said chimeric decoy oligonucleotide is SEQ ID NO:8.

20. The chimeric decoy oligonucleotide of claim 18, wherein said chimeric decoy oligonucleotide is SEQ ID NO:9.

21. The chimeric decoy oligonucleotide of claim 18, wherein said chimeric decoy oligonucleotide is SEQ ID NO:10.

22. The chimeric decoy oligonucleotide of claim 18, wherein said chimeric decoy oligonucleotide is SEQ ID NO:11.

23. A method of decreasing the metastatic ability of a cancer cell, comprising the step of
introducing into said cancer cell a chimeric decoy oligonucleotide, wherein said chimeric decoy oligonucleotide binds at least two eukaryotic transcription factors, and wherein said at least two eukaryotic transcription factors bind to a promoter for a gene encoding an MMP.
